(19) United States
(12) Patent Application Publication
(10) Pub. No.: US 2013/0197353 A1
(43) Pub. Date: Aug. 1, 2013

(54) RADIOPAQUE MARKER FOR A CATHETER

(71) Applicant: Randolf Von Oepen, Aptos, CA (US)

(72) Inventor: Randolf Von Oepen, Aptos, CA (US)

(21) Appl. No.: 13/359,694

(22) Filed: Jan. 27, 2012

(51) Int. Cl. A61B 5/05; B23K 26/00; C23F 1/00; B26D 3/11

(52) U.S. Cl. 600/424; 29/2.25; 219/121.72; 216/58

(57) ABSTRACT

Radiopaque marker for a catheter includes a collar having a ring shape defining a longitudinal center axis therethrough and at least one helical member extending longitudinally from the collar. A second collar can be provided spaced longitudinally from the first collar. Additionally, a second helical member can extend longitudinally from the collar and be coaxially intertwined with the first helical member in a double helical configuration. The helical member can define a compression spring structure having longitudinal, transverse, and torsional flexibility. A catheter having such a marker and a method of making the marker are also provided.

### BACKGROUND OF THE DISCLOSED SUBJECT MATTER

1. Field of the Disclosed Subject Matter

The disclosed subject matter relates to a radiopaque marker, a catheter with a radiopaque marker, and a method of manufacturing the same. More particularly, the presently disclosed subject matter relates to a radiopaque marker including a collar and at least one helical member extending from the collar.

2. Description of Related Subject Matter

Cardiovascular disease is prevalent in the United States and in other parts of the world. One manifestation of cardiovascular disease is atherosclerosis, which is the buildup of plaque (or fatty deposits) on the walls of blood vessels, such as coronary and peripheral arteries. This buildup of plaque can grow large enough to reduce or occlude blood flow through the blood vessel. Serious damage results when an area of plaque ruptures from the vessel wall and forms a clot, which can travel to another part of the body. If the blood vessels that feed the heart are blocked, a heart attack can result. If the blood vessels to the brain are blocked, a stroke can result. Thus, atherosclerosis can be fatal.

Typically, physicians treat atherosclerosis by implanting a tubular endoprosthesis such as a stent at the narrowed or blocked segment of the blood vessel. The endoprosthesis when expanded widens and holds open the blood vessel. To perform this procedure, the stent is delivered to the site of the lesion in the blood vessel by a catheter assembly, otherwise known as a stent delivery device. The stent delivery device enters the vasculature of the patient through the femoral artery and travels through a tortuous path to the site of the lesion. The physician positions the stent across the lesion and deploys the stent to expand the stent and force the plaque against the inside wall of the blood vessel (or lumen). The stent maintains its expanded configuration to maintain the patency of the blood vessel is maintained.

Typical stent delivery systems include radiopaque marker bands to allow for visualization under fluoroscopy so that the physician can see when the stent is positioned accurately across the lesion. Radiopaque markers are commonly formed from a rigid metal with adequate radiopacity, for example but not limited to, platinum and/or iridium. A known stent delivery device, such as that described in PCT Publication Number WO 98/07390, has short solid tubular bands made from a radiopaque material. Due to their rigidity and length, conventional marker bands can increase the profile of the balloon and create an abrupt stiffness transition point in the catheter. This can result in a reduction in flexibility of the catheter tube. The transition point can decrease pushability by allowing buckling or kinking and thus hinder tracking through the vasculature by inhibiting the shape of the catheter to conform to the anatomy. Therefore, the known catheter, when pushed forward through a curved vessel, suffers from undesired stiffness in the region of the marker bands; diminishing especially the trackability of the catheter and resulting in kinking and buckling of the catheter.

As an alternative, radiopaque coil markers have been developed to increase flexibility of the marker. Radiopaque coil markers present challenges in manufacture and design. For example, coil markers can be difficult to mount securely on a catheter member. Similarly, forming flexible markers from cylindrical tubes or the like has been heretofore impractical. For example, the removal of slag resulting from conventional lasers is extremely difficult or impossible because of the fragility of the small markers. Thus, handling the markers often causes damage.

Thus, for at least the aforementioned reasons, there is a need to improve marker flexibility, improve catheter deliverability, provide greater marker trackability, improve pushability, and reduce the likelihood of kinking and bulking of radiopaque markers with catheters.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve the above and other advantages and in accordance with the purpose of the disclosed subject matter, as embodied and broadly described, the disclosed subject matter provides a radiopaque marker for a catheter, comprising a collar having a ring shape defining a longitudinal center axis therethrough and at least one helical member extending longitudinally from the collar.

The disclosed subject matter also includes a catheter comprising an elongate tubular member having a lumen defined at least partially therethrough; and a radiopaque marker coupled to the elongate tubular member. The radiopaque marker comprises a collar having a ring shape defining a longitudinal center axis therethrough, and at least one helical member extending longitudinally from the collar.

Further in accordance with the disclosed subject matter, there is provided a method of manufacturing a radiopaque marker for a catheter, comprising providing an elongate tube of material having a lumen extending longitudinally therethrough; and cutting a portion of the tube to define a collar and at least one helical member extending longitudinally from the collar.

It is to be understood that both the foregoing general description and the following detailed description are embodiments and are intended to provide further explanation of the disclosed subject matter claimed. The accompanying drawings, which are incorporated in and constitute part of this specification, are included to illustrate and provide a further understanding of the system and method of the disclosed subject matter. Together with the description, the drawings serve to explain the principles of the disclosed subject matter.

### BRIEF DESCRIPTION OF THE DRAWINGS

The following is a description of some specific embodiments of the disclosed subject matter, reference being made to the accompanying drawings, in which:

FIG. 1 is a schematic side view of a representative embodiment of a radiopaque marker mounted on a catheter shaft in accordance with the disclosed subject matter;

FIG. 2A is a detailed view of a collar of the radiopaque marker of FIG. 1, and FIG. 2B is a cross-section of the collar along the lines B-B of FIG. 2A, in accordance with an embodiment of the disclosed subject matter;

FIG. 3 is a perspective view of a radiopaque marker with a double helical configuration, in accordance with an alternative embodiment of the disclosed subject matter;

FIG. 4A is a perspective view of tubular stock material to construct the radiopaque marker in accordance with an embodiment of the disclosed subject matter, and FIG. 4B is a schematic depiction of a process of making the radiopaque marker in accordance with an embodiment of the disclosed subject matter;

FIG. 5 is a partial cross section view of the distal end portion of a balloon catheter provided with radiopaque markers in accordance with an embodiment of the disclosed subject matter;

FIG. 6 is a side view of a conventional marker coupled to a catheter member; and

FIG. 7 is a side view of a representative embodiment of a radiopaque marker coupled to a tubular member in accordance with an embodiment of the disclosed subject matter for purpose of comparison with FIG. 6.

While the subject matter is capable of various modifications and alternative forms, specific embodiments thereof are depicted in the drawings, and will herein be described in detail for the purpose of illustration and not limitation. It should be understood, however, that it is not intended to limit the subject matter to the particular forms disclosed but, to the contrary, the intention is to cover all modifications, equivalents, and alternatives falling within the spirit and scope of the subject matter as defined by the appended claims. It will be apparent to those skilled in the art that various modifications and variations can be made to the radiopaque marker and catheter without departing from the spirit or scope of the subject matter. Thus, it is intended that the disclosed subject matter include modifications and variations that are within the scope of the appended claims and their equivalents.

### DETAILED DESCRIPTION

While the disclosed subject matter herein can be embodied in many different forms, reference will now be made in detail to specific embodiments, examples of which are illustrated in the accompanying drawings. For the purposes of this disclosure, like reference numbers in the figures shall refer to like features unless otherwise indicated.

The disclosed subject matter provides a radiopaque marker, a method of making the same, and a catheter mounting the radiopaque marker. The disclosed subject matter provides a radiopaque marker with improved visualization under fluoroscopic equipment as well as a greater flexibility. The radiopaque marker according to the subject matter, includes a collar having a ring shape defining a longitudinal center axis therethrough; and at least one helical member extending longitudinally from the collar.

In accordance with another aspect of the disclosed subject matter, a method of manufacturing a radiopaque marker for a catheter is also provided. The method includes providing an elongate tube having a lumen extending longitudinally therethrough; and cutting a portion of the tube to define a collar and at least one helical member extending longitudinally from the collar.

The disclosed subject matter also includes a catheter with the radiopaque marker as disclosed. The catheter includes an elongate tubular member having a lumen at least partially defined therethrough and a radiopaque marker coupled to the elongate tubular member. The radiopaque marker comprises a collar having a ring shape defining a longitudinal center axis therethrough and at least one helical member extending longitudinally from the collar.

The catheter with the radiopaque marker as disclosed provides improved trackability of the catheter, improved pushability, and reduction in the likelihood of kinking and bulking of the catheter at the radiopaque marker. The catheter having the radiopaque marker can be configured for a variety of known interventional techniques, such as but not limited to, delivering a stent or the like.

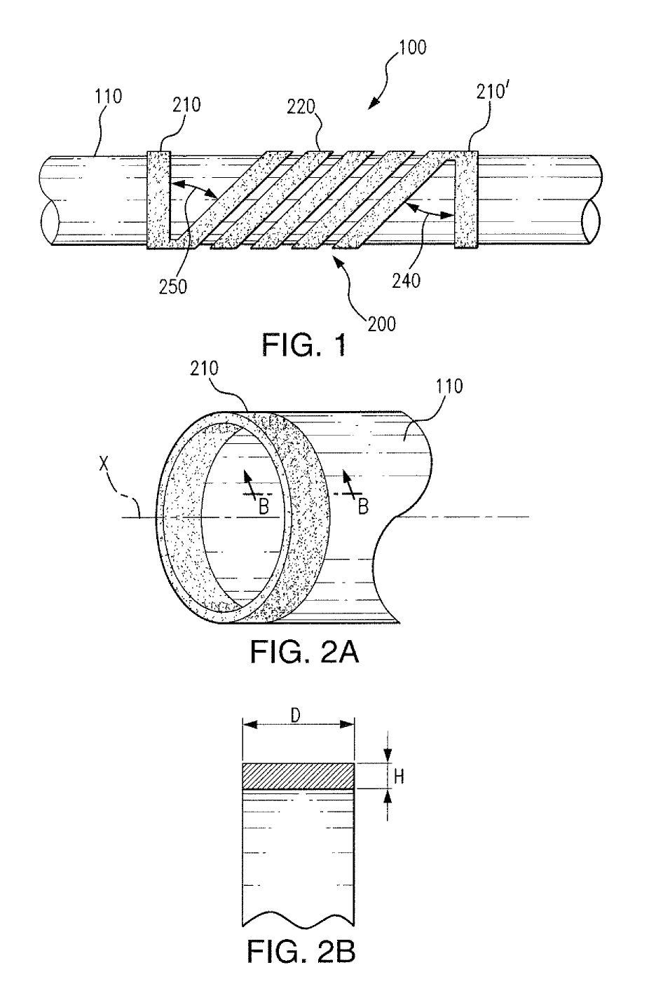

For purpose of illustration, and not limitation, reference is now made to a representative embodiment of a radiopaque marker of the disclosed subject matter. As shown in FIG. 1, the radiopaque marker embodied herein, generally referenced as 200, includes a collar 210 and at least one helical member 220 extending longitudinally from the collar 210. As depicted, the collar and the helical member are formed as a monolithic structure and more particularly from a single piece of material.

In accordance with another aspect of the disclosed subject matter, the radiopaque marker can include a second collar spaced from the first collar. For example, the radiopaque marker 200 of FIG. 1 has a first collar 210 and also a second collar 210′. The helical member 220 extends longitudinally between the first collar 210 and the second collar 210′. Each collar can define an opposing end of the radiopaque marker, respectively.

As depicted in FIG. 1, the collar(s) and the helical member define a substantially cylindrical structure. The radiopaque marker can have any suitable inner diameter and outer diameter. By forming the radiopaque marker as a monolithic structure, the collar and the helical member can have the same inner diameters and outer diameters, respectively. For example, and not limitation, the inner diameter of the collar and of the helical member for a radiopaque marker for a coronary balloon catheter generally would be at least about 0.4 mm and for a peripheral balloon catheter generally would be at least about 0.2 mm, and can range up to 1.1 mm for coronary balloon catheters and for peripheral balloon catheters. Likewise, for purpose of illustration, the overall length of the radiopaque marker 200 for a coronary balloon catheter can range from approximately from 0.4 mm to 2.0 mm. Other lengths are contemplated and will depend upon the intended purpose. Due to the increased flexibility of the radiopaque marker, lengths at least up to 200 mm can be used.

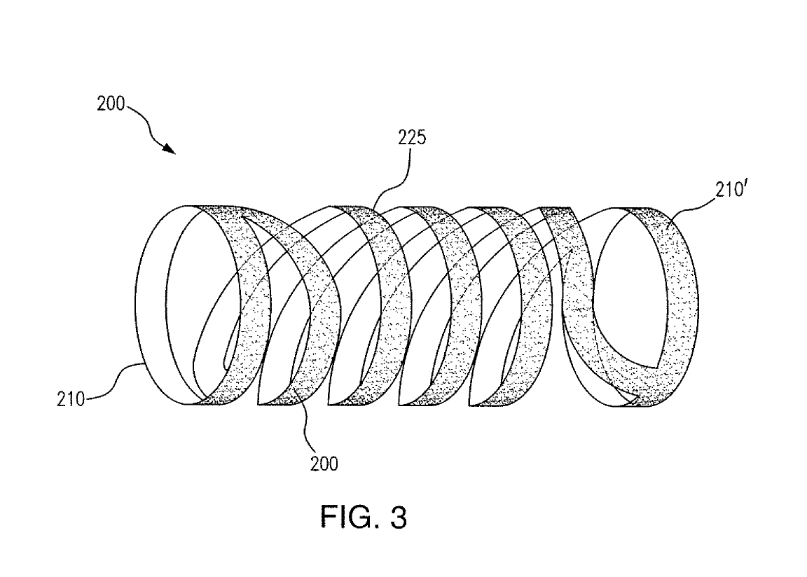

The collar 210 of the disclosed subject matter generally has a ring shape defining a longitudinal center axis X therethrough. FIG. 2A provides a detailed view of the collar 210. The collar 210, as depicted, is a closed ring, although an open ring shape can be used as needed. By providing a closed or substantially closed ring configuration, the collar can be mounted securely to a shaft member of the catheter, such as by swaging or the like.

FIG. 2B shows the cross section of the collar of FIG. 2A along the line B-B. In the depicted embodiment, the collar 210 has a generally rectangular cross section with a width D and a thickness H. The cross section of the collar, however, can be different shapes and not limited to rectangular. For example, the radiopaque marker can be processed to provide a collar having a trapazoidal, triangular or rounded cross-section.

Similarly, the helical member 220 can likewise have a generally rectangular cross section with a width D and thickness H. The helical member 220 and/or collar 210 can have a ratio of width to thickness of at least about 1:1 and thus have a generally square cross section or the width and thickness can differ. Additionally or alternatively, either or both the width and/or the thickness of the helical member can vary longitudinally along the length of the helical member to achieve desired flexibility and kink resistance. For example, the helical member can be provided with a trapezoid configuration or an alternating cross-section of undulating width or thickness.

The helical member 220 extends from the collar 210 at an angle 240, as depicted in FIG. 1. The helical member extends from the second collar 210′, if provided, at an angle 250. For example, the angle between the helical member 220 and the collars 210, 210′ can be acute, e.g., about 45°. In another embodiment, the angle 240 and angle 250 are different angles.

The helical member 220 can have a constant pitch along the length of the radiopaque marker, or the helical member can have varying pitch along at least a length of the radiopaque marker. The helical member 220 can have one or a plurality of pitches longitudinally along the length of the helical member. For example, the helical member 220 can include a first configuration having a first pitch extending from the collar 210 and a second configuration having a second pitch extending from the second collar 210′. In this manner, a plurality of pitches can define a stiffness gradient along the length of the radiopaque marker 200. Sections with greater density and tight or decreased pitches along the tubular member 110 have greater visual contrasts under X-ray fluoroscopy. Thus, such sections exhibit varied radiopacity and pushability.

As such, and as embodied herein, the helical member 220 can define a compression spring-like structure with longitudinal, transverse, and torsional flexibility. Such flexibility can reduce the likelihood of kinking and bulking of the radiopaque marker with a tubular member of a catheter. The monolithic radiopaque marker as disclosed herein can be further modified to incorporate the various configurations and advantages of a coil-type marker as disclosed in U.S. Ser. No. 11/775,480, the contents of which are incorporated by reference herein.

In further embodiment as provided in FIG. 3, the radiopaque marker 200 can include a second helical member 225 extending longitudinally from the collar 210. The second helical member 225 is coaxially intertwined with the first helical member 220 in a double helical configuration, as depicted in FIG. 3. As embodied herein, the radiopaque marker 200 is depicted with two collars 210, 210′. Based on the configuration, the double helical members can increase or decrease flexibility depending on the desired use.

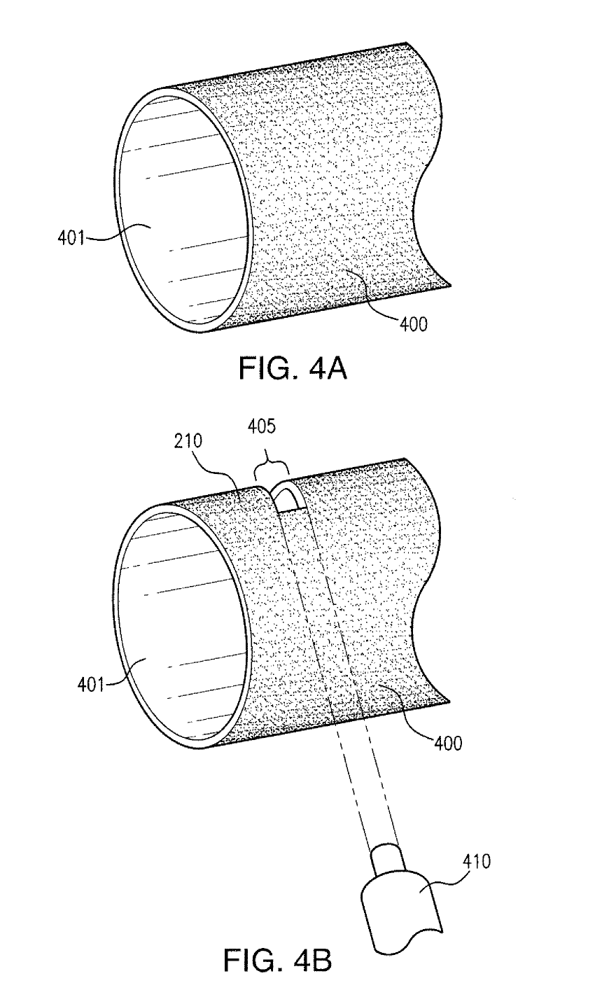

In accordance with another aspect of the disclosed subject matter, a method is provided for making the radiopaque marker 200, including providing an elongate tube of material having a lumen extending longitudinally therethrough and cutting a portion of the tube to define a collar and at least one helical member extending longitudinally from the collar. FIG. 4A depicts an elongate tube of material 400 having a lumen 401 extending longitudinally therethrough. FIG. 4B depicts a portion 405 of the tube cut to define the collar 210 and the helical member 220 extending longitudinally from the collar 210.

Cutting can be performed using a variety of known techniques. For example, and not with limitations, cutting can be performed by a laser 410. To reduce the disadvantages of conventional lasers, such as slag, the laser used herein performs a short-pulse laser process, such as pico-second or femtosecond laser cutting process. Other laser processes currently in development with even shorter pulse duration, such as, for example an atom second laser, are further contemplated herein as well. Alternative or in addition to laser processing, etching can be used to manufacture the radiopaque marker. Other methods to cut the structure of the radiopaque marker are contemplated here, such as, but not limited to, using sharp cutting tools like a razer blade, Such processes provide exceptional surface finishing which can reduce the need for post-process polishing. However, the method can also include polishing the collar and the helical member, as needed or desired. Methods for polishing the radiopaque marker can include, but are not limited to, electropolishing, blasting, tumbling, chemical etching or other suitable methods to remove material and reduce sharp edges. In this regard, the disclosed subject matter includes making a radiopaque marker using laser techniques without the drawbacks of significant slag that is difficult or impossible to remove without damage to the radiopaque marker.

The elongate tube of material 400 can be radiopaque material. An example of such radiopaque material includes, but is not limited to, platinum, tantalum, tungsten, silver, gold, niobium, barium sulfate, iodine, other suitable materials with a high density polymer, or alloy or combination thereof. United States Publication Number 2011/0070355 to Bavaro et al. and U.S. Pat. Nos. 7,303,798 to Bavaro et al., 7,322,959 to Warnack at al., and 7,833,597 to Bavaro et al. discuss various radiopaque materials and radiopaque markers and the disclosures of which are herein incorporated by reference in their entirety.

Alternatively or additionally, the marker can be coated with a radiopaque material. For example, if the elongate tube 400 is not radiopaque, the method further includes coating the elongate tube with radiopaque material, such as, after cutting is performed. The coating can be achieved in a variety of ways. For example, the covering can be formed from a coating sprayed, sputtered, dipped or otherwise layered on the exterior surface of the marker. Additional coatings also can be provided as desired or needed.

The radiopaque marker 200 can be affixed to an elongated tubular shaft of a catheter 110 in a number of ways. For example, a collar alone can couple the radiopaque marker 200 to the tubular member having the helical member 220 unsecured to the tubular member. In another embodiment, both the collar 210 and the helical member 220 are secured to the tubular member. Likewise, the radiopaque marker can be attached to either the inner wall or outer wall surface at the catheter shaft, and can be located anywhere along the length of the shaft as desired.

In one embodiment, the collar 210 is at least partially embedded in the wall of the tubular member 110. For example, the collar can be swaged or pressure fit at least partially into the wall of the tubular member 110. The collar provides full circumferential contact with the wall of the tubular member and eliminates any exposed sharp edges. The collar allows the ring to be more easily bonded to the shaft member, such as within the inner guidewire lumen, if desired, or an outer surface of the inner tubular member of a catheter. Other known techniques, such as securing the marker by adhesives, thermal or mechanical bonds, are herewith contemplated. Furthermore, techniques such as applying heat to the marker and melting the marker to a member, such as, a member such as a polymeric tube. Further, the radiopaque marker can be adhered to the surface of the tubular member. The catheter can further include a cover disposed over the marker. The cover can be a coating, layer, or membrane over the marker or a tubular member, such as a shrink wrap material, disposed on the exposed surface of the marker.

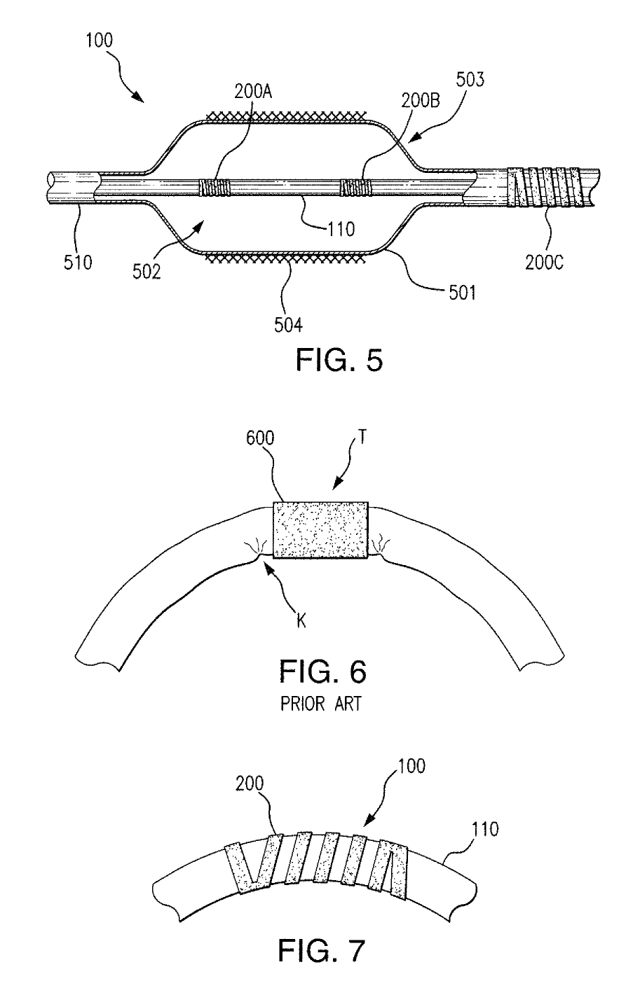

The catheter 100 can further include a balloon 501, as depicted in FIG. 5. The balloon 501 can also have a stent 504 positioned on an exterior surface of the balloon 501. In accordance with one embodiment of the disclosed subject matter, the balloon 501 is sealingly coupled to an outer tube 510 of the catheter. The balloon 501 defines an interior volume in communication with an inflation lumen of the catheter. As such, for example, a fluid can be introduced through the proximal end of the lumen to pressurize the balloon 501 and display the stent mounted thereon. Tubular member 110 is disposed concentrically within the balloon 501 in the embodiment of FIG. 5. The tubular member 110 can define a second inner lumen for a guidewire.

The radiopaque marker can be coupled to the tubular member 110 within the balloon 501 between a first end 502 and second end 503 of the balloon. For purpose of illustration and not limitation, FIG. 5 provides a first radiopaque marker 200A with two collars located near the first end 502 and a second radiopaque marker 200B with one collar located near the second end 503 of the balloon. Additionally or alternatively, the radiopaque marker can be positioned outside the boundary of the balloon, such as for example, but not limited to, near the tip of the catheter 100. Radiopaque marker 200C is located near the tip of the catheter 100, as provided in FIG. 5.

In another embodiment, the radiopaque marker disclosed herein can be used on a catheter for delivery of a self-expanding device, such as a stent or filter. In this regard, the radiopaque marker can be positioned at a plurality of positions including at either or both ends of the stent seat of the catheter. Regardless of the type of delivery device of the catheter, the elongate tubular member can be disposed within an outer tube defining a guidewire lumen. In yet another example, the catheter can be an angioplasty catheter, rather than a stent delivery device. In this regard, the angioplasty catheter can include a balloon having a drug coating, if desired. It should be recognized that the tubular member of the disclosed subject matter can be utilized in any of a variety of interventional delivery devices or other catheter products. Further, the catheter can be any of a variety of known types, including a coaxial catheter configuration or a dual liner catheter configuration as well as over the wire or rapid exchange configuration. The disclosed subject matter is not hereby limited.

FIG. 6 depicts a known conventional marker 600 on a catheter in a bent state. Known radiopaque markers suffer from a stiff transition point T caused by the marker attached to the catheter tubing. In particular, the known markers additionally provide a kinking point K along the catheter shaft which is undesirable.

By contrast, FIG. 7 depicts for purpose of illustration and not limitation certain improvements provided by the radiopaque marker of the disclosed subject matter. As noted above, the marker 200 is a monolithic structure having a ring-type collar capable of secure engagement with the wall of a catheter shaft or the like. Furthermore, the marker 200 of the disclosed subject matter provides a smooth flexible transition and reduces or prevents kinking of the catheter in the depicted bent state. The inner radius of the marker along the bend is compressed in this state while the outer radius of the marker is expanded thus ensuring a higher flexibility and trackability of the catheter. The marker eliminates the stiff transition points and kinking points that are generally found with known markers. The gradual stiffness transition of the radiopaque marker 200 with the catheter member 110 also helps support the column strength of the overall catheter 100.

The radiopaque markers of the disclosed subject matter reduces the rigid length of the markers as well as reduces the profile of the markers. Therefore, the catheter according to embodiments of the disclosed subject matter provides a greater flexibility and trackability, while providing an indication of a selected length of the catheter (e.g. the balloon) and/or the length of a device mounted on the catheter.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

Many modifications, variations, or other equivalents to the specific embodiments described above will be apparent to those familiar with the art. It is intended that the scope of this disclosed subject matter be defined by the claims below and those modifications, variations and equivalents apparent to practitioners familiar with this art.

What is claimed is:

1. A radiopaque marker for a catheter, comprising:
a collar having a ring shape defining a longitudinal center axis therethrough; and
at least one helical member extending longitudinally from the collar.

2. The radiopaque marker of claim 1, wherein the collar is a closed ring.

3. The radiopaque marker of claim 1, wherein the collar has a generally rectangular cross section.

4. The radiopaque marker of claim 1, wherein the helical member has a generally rectangular cross section with a width and a thickness.

5. The radiopaque marker of claim 4, wherein the helical member has a ratio of width to thickness of at least about 1:1.

6. The radiopaque marker of claim 4, wherein the width of the helical member varies longitudinally along its length.

7. The radiopaque marker of claim 4, wherein the thickness of the helical member varies longitudinally along its length.

8. The radiopaque marker of claim 1, wherein the helical member has a constant pitch longitudinally along its length.

9. The radiopaque marker of claim 1, wherein the helical member has a varied pitch longitudinally along its length.

10. The radiopaque marker of claim 1, wherein the helical member defines a compression spring structure having longitudinal, transverse, and torsional flexibility.

11. The radiopaque marker of claim 1, further comprising a second helical member extending longitudinally from the collar and coaxially intertwined with the first helical member in a double helical configuration.

12. The radiopaque marker of claim 1, wherein the collar and the helical member define a substantially cylindrical structure.

13. The radiopaque marker of claim 1, wherein the collar and the helical member are a monolithic structure.

14. The radiopaque marker of claim 1, wherein the radiopaque marker has a length approximately between about 0.4 mm to about 2.0 mm.

15. The radiopaque marker of claim 1, wherein the radiopaque marker has an inner diameter of at least about 0.2 mm.

16. The radiopaque marker of claim 1, wherein the radiopaque marker is formed of a material selected from at least one of platinum, tantalum, gold, tungsten, silver, niobium, barium sulfate, iodine, or a high density polymeror alloy, or combination thereof.

17. The radiopaque marker of claim 1, further comprising a second collar at an opposite longitudinal end of the helical member from the first collar.

18. A catheter comprising:
an elongate tubular member having a lumen defined at least partially therethrough; and
a radiopaque marker coupled to the elongate tubular member, the radiopaque marker comprising
a collar having a ring shape defining a longitudinal center axis therethrough, and
at least one helical member extending longitudinally from the collar.

19. The catheter of claim 18, wherein the collar is at least partially embedded in a wall of the elongate tubular member.

20. The catheter of claim 18, wherein the collar is one of melted by application of heat, swaged, or press-fit into the wall of the elongate tubular member.

21. The catheter of claim 18, further comprising a balloon disposed at a distal end of the elongate tubular member.

22. The catheter according to claim 21, wherein the balloon has a first end and a second end with the elongated tubular member extending therethrough, and further wherein the radiopaque marker is coupled to the elongate tubular member within the balloon between the first end and the second end thereof.

23. A method of manufacturing a radiopaque marker for a catheter, comprising:
providing an elongate tube of material having a lumen extending longitudinally therethrough; and
cutting a portion of the tube to define a collar and at least one helical member extending longitudinally from the collar.

24. The method of claim 23, wherein cutting is performed by a laser.

25. The method of claim 24, wherein the laser performs a short-pulse laser process.

26. The method of claim 25, wherein the short-pulse laser process is one of a pico-second cutting process, an atom-second cutting process, or a femto-cutting process.

27. The method of claim 23, further comprising polishing the collar and the helical member.

28. The method of claim 27, wherein the polishing includes at least one of electro polishing, blasting, tumbling, or chemical etching.

29. The method of claim 23, wherein the cutting comprises an etching process.

30. The method of claim 23, further comprising affixing the radiopaque marker to an elongated tubular shaft of a catheter.

31. The method of claim 30, wherein the affixing includes swaging the radiopaque marker in a wall of the elongated tubular shaft.

32. The method of claim 23, wherein the elongate tube is made of radiopaque material.

33. The method of claim 23, further comprising coating the elongate tube with radiopaque material.
